(19) United States
(12) Plant Patent Application Publication
(10) Pub. No.: US 2014/0041090 P1
(43) Pub. Date: Feb. 6, 2014

(54) Variety of calla lily named 'Red Alert'

(50) ;

(71) Applicant: Cecilius Jan-Jochem Randag, 't Zand (NL)

(72) Inventor: Cecilius Jan-Jochem Randag, 't Zand (NL)

(73) Assignee: SANDE BV, 't Zand (NL)

(21) Appl. No.: 13/507,856

(22) Filed: Aug. 2, 2012

(51) Int. Cl. A01H 5/00

(52) U.S. Cl. PLT/490

(57) ABSTRACT

‘Red Alert’ is a new variety of calla lily having flowers with a reddish orange-colored spathe that produces 2-7 flowers per tuber. The flower stems are between 20-30 cm. in height and the leaves are light green with whitish spots and have a leathery texture.

### BOTANICAL CLASSIFICATION

Zantedeschia sprengeri

### VARIETAL DENOMINATION

‘RED ALERT’

### BACKGROUND OF THE NEW PLANT

The present invention comprises a new and distinct cultivar of calla lily, botanically known as Zantedeschia sprengeri and hereinafter referred to by the cultivar name ‘Red Alert’.

‘Red Alert’ is a product of a planned breeding program that had the objective of creating Zantedeschia hybrids for cut flower production in a wide range of colors with a large, classic flower shape. The breeding program began in 1989, and the new cultivar is a seedling selected from the crossing of Zantedeschia sprengeri variety ‘Hot Shot’ (female parent, unpatented) with Zantedeschia sprengeri C02028 (male parent, unpatented). ‘Red Alert’ was selected in 2004 by the inventor C. J. J. Randag in 't Zand, The Netherlands as one flowering plant within the progeny of the stated cross.

The first act of asexual reproduction of ‘Red Alert’ by tissue culture was performed by the inventor in August of 2006 in 't Zand, The Netherlands. Further asexual reproductions by tissue culture at the same location has demonstrated that the combination of characteristics as herein disclosed for the new cultivar are retained and reproduced true to type through successive generations of asexual reproduction.

The following observations, measurements and comparisons describe plants grown in 14 cm. pots in 't Zand, The Netherlands under greenhouse conditions which approximate those generally used in horticultural practice. Color references are made to the R.H.S. Colour Chart of The Royal Horticulture Society of London, except where general color terms of ordinary significance are used.

The present invention has not been evaluated under all possible environmental conditions. The phenotype may vary with variation in environment without a change in the genotype of the plant.

The following traits have been repeatedly observed and determined to basic characteristics of ‘Red Alert’ which, in combination, distinguish this calla lily as a new and distinct cultivar:

1. Reddish orange-colored spathe;

2. Fast forcing time; and

3. Large round flowers.

Table 1 provides similarities and differences of ‘Red Alert’ to its parents and Zantedeschia sprengeri ‘Mango’ (unpatented).

TABLE 1

| ‘Red Alert’ | Similarities | Differences |
|---|---|---|
| ‘Hot Shot’ | Leaf shape | Shorter plant height |
| C02028 | Number of | Broader leaves |
| | spots on leaves | Reddish orange color |
| ‘Mango’ | Leaf shape | Plant height |
| | Spathe color | |

### BRIEF DESCRIPTION OF THE DRAWINGS

The accompanying photographic drawings illustrate the new variety, with the colors being as nearly true as is possible with color illustrations of this type:

FIG. 1 is a close-up photograph of the new variety; and

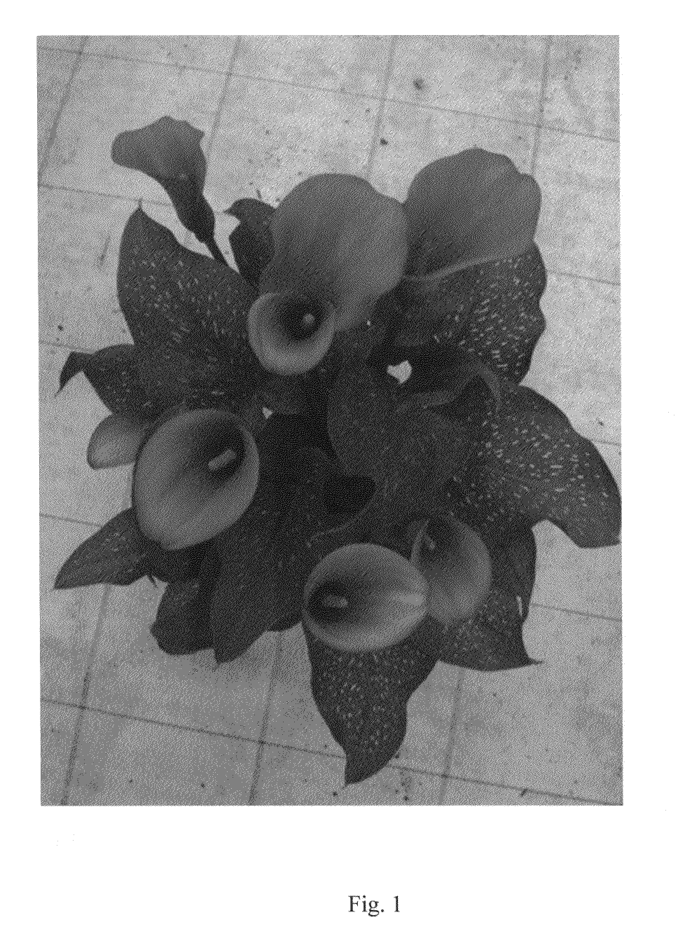

FIG. 2 is a photograph of multiple plants of the new variety.

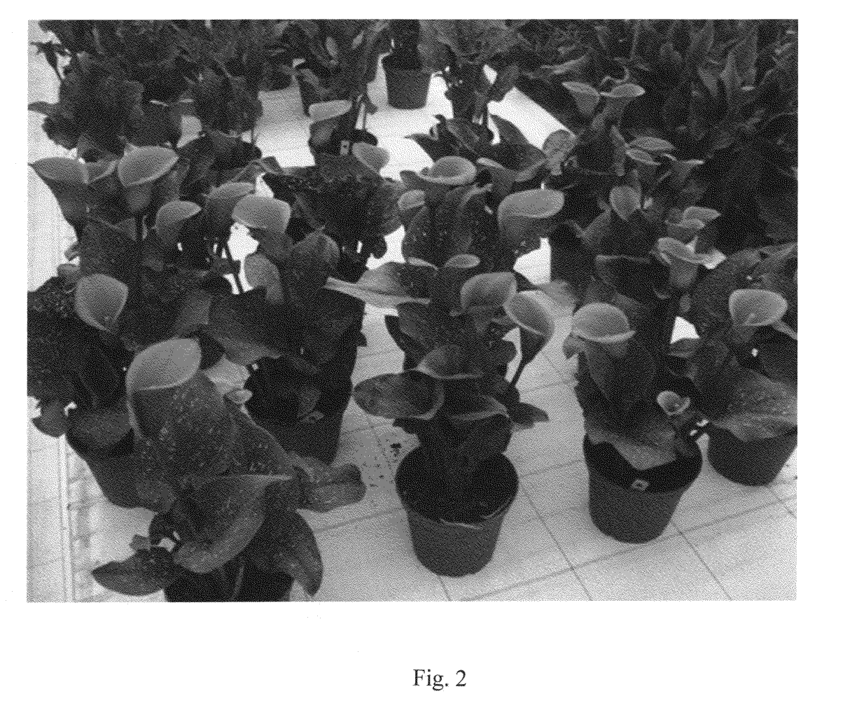

### DESCRIPTION OF THE PLANT

The plant:
   Size.—Height of the leaf canopy above the soil: 20-25 cm. Height of top of flower above the soil: 35-40 cm. Diameter: 25-30 cm.
   Form.—Erect.
   Number of flowers per tuber.—Size 9-12 cm. in diameter: 1-3 flowers. Size 12-15 cm. in diameter: 2-4 flowers. Size 15-18 cm. in diameter: 3-8 flowers.
   Branches.—Character: Like the variety ‘Picasso’ (U.S. Plant Pat. No 15,282). Color: Between Yellow-Green Group RHS 145A and 145B, with spots between White Group 155A and N155A.
   Leaves.—Size: Width: 5-12 cm. Length: 20-35 cm. Number per plant: 10-30. Shape: Cordate. Color: Upper surface: Yellow-Green Group RHS 146B to 147A. Lower surface: Yellow-Green Group RHS 146B to 147A. Margin: Wavy. Veins: Configuration: Pinnate. Color: Yellow-Green Group RHS 147B. Surface quality: Leathery. Petiole: Length: 50-65 cm. Color: Between Yellow-Green Group RHS 146B to 147A.
   Roots.—Color: White. Branching: Similar to other Zantedeschia varieties.
The flower and seeds:
   Spathe.—Size: Length: 6-12 cm. Width: 4-9 cm. Color: Upper surface: Varies between Red Group RHS 44B and 46A. Lower surface: Varies between Red Group RHS 44B and 46A; a very small light pink blush sometimes appears on the edge of the spathe when maturing. Vein color: Approximately Red Group RHS 53A. Shape: Cupped.
   Spadix.—Size: Length: 10-25 mm. Diameter: 2-4 mm. Color: Between Yellow-Orange Group RHS 16A and 16C, with Red-Purple Group RHS 60A spots on the upper portion. Position relative to spathe: Upright.
   Peduncle.—Size: Length: 20-30 cm. Diameter: 5-10 mm. Color: Upper part varies between Yellow-Green Group RHS 144A and N144A, with spots between Red-Purple Group RHS 59A and 60A. The lower part is Green-White Group RHS 157C.
   Reproductive organs.—Location of female organs: Basal position of the spadix. Location of male organs: Upper position of the spadix. Perianth: Conspicuous. Stamens: Presence: Not visible before pollen release. Number: More than 50. Pollen amount: Similar to ‘Picasso’. Color: Between White Group RHS 155A and 155B. Anther: Shape: Round and wire-like. Length: Less than 1 mm. Color: Between White Group RHS 155A and 155B. Filament: Length: Less than 1 mm. Color: Between White Group RHS 155A and 155B. Pistil: Number: 10 to 50. Length beyond perianth: About 1 mm. Stigma: Shape: Round. Size: Less than 1 mm. Style: Length: Less than 1 mm. Color: Between Red-Purple Group RHS 59A and 60A. Ovaries: Texture: Smooth. Length: Between 1 to 10 mm. Width: Between 1 to 10 mm. Color: Between Yellow-Green Group RHS 144B and 144C, with portions of Red-Purple Group RHS 59A and 60A.
   Seeds.—Length: 4-7 mm. Width: 3-6 mm. Color: Between Yellow-Green Group RHS 150A and 151A. Amount: Between 1-100.
General:
   Disease resistance.—No unusual susceptibility to diseases noticed to date.
   Pest resistance.—No unusual susceptibility to pests noticed to date.
   Heat tolerance.—No heat tolerance.
   Hardiness.—Not hardy.
   Flowering.—Tubers planted in a greenhouse in The Netherlands in February; flowers produced in April. Tubers planted outdoors in The Netherlands in May; flowers produced in July.
   Lastingness.—About two weeks on the plant.
   Fragrance.—None.

I claim:

1. A new and distinct variety of calla lily plant named ‘Red Alert’ as herein described and illustrated.
